(19) United States
(12) Patent Application Publication
(10) Pub. No.: US 2003/0018123 A1
(43) Pub. Date: Jan. 23, 2003

(54) Biocompatible optically transparent polymeric material

(75) Inventors: Sergei Nikolaevich Bagrov, Moscow (RU); Galina Dmitrievna Leontieva, Moscow (RU); Sergei Viktorovich Novikov, Moscow (RU); Irina Alexandrovna Maklakova, Moscow (RU)

Correspondence Address: LADAS & PARRY, 26 WEST 61ST STREET, NEW YORK, NY, 10023, US

(73) Assignee: OOO " NAUCHNO-EXPERIMENTALNOE PROIZVODSTVO MIKROKHIRURGIYA GLAZA "

(21) Appl. No.: 10/184,121

(22) Filed: Jun. 27, 2002

(30) Foreign Application Priority Data
Jul. 4, 2001 (RU) 2001118087

(51) Int. Cl. A61F 2/00; C08G 63/91

(52) U.S. Cl. 525/54.11; 424/423

(57) ABSTRACT

A biocompatible optically transparent polymeric material is proposed, which has high biocompatibility, transparency in the visible region, hydrophily, elasticity and mechanical strength, and has a spectrum cutting off the UV band, the material being designed for the production of intraocular implants and contact lenses.

### FIELD OF THE INVENTION

The invention relates to medicine, and more concretely to ophthalmology, and is designed for the production of intraocular implants, in particular intraocular lenses (IOL), and also contact lenses.

### BACKGROUND OF THE INVENTION

A biocompatible optically transparent polymeric material is known, comprising collagen, water-soluble ethylenically unsaturated compounds and a cross-linking agent, which material is used for the production of intraocular lenses (U.S. Pat. No. 5,993,796).

A drawback of this material is the optical passage of wavelengths in the UV region of the spectrum. UV radiation has a negative effect on the retina of the eye.

The object of the invention is to create a biocompatible optically transparent polymeric material, which has high biocompatibility, transparency in the visible region, hydrophily, elasticity and mechanical strength, and has a spectrum Cutting off the UV band.

### DETAILED DESCRIPTION OF THE INVENTION

The technical result is achieved in that a biocompatible, optically transparent polymeric material, comprising collagen, at least one water-soluble ethylenically unsaturated compound and a cross-linking agent, in accordance with the invention additionally comprises at least one UV adsorber having at least one functional group with the following relationship of the components (wt. %):

| | |
|---|---|
| Collagen | 0.00001-20 |
| Ethylenically unsaturated compound | 10-99.99899 |
| Cross-linking agent | 0-10 |
| UV adsorber | 0.001-10 |
| Water | balance to 100. |

Highly purified collagen from connective tissue is used as the collagen.

The concentration of the collagen cannot be less than 0.00001% because of the marked reduction of the biocompatibility of the material at lower values and cannot be more than 20% because of a reduction of the strength and optical characteristics at higher values.

Acrylic, vinyl and other monomers are used as the ethylenlically unsaturated compounds. It is more preferable to use vinylpyrrolidone and derivatives thereof, alkyl acrylates, hydroxy alkyl acrylates, acrylamide vinylpyrrolidone and derivatives thereof, etc.

The concentration of the ethylenically unsaturated compound cannot be less than 10% because of reduction of the strength characteristics of the material at lower values.

Any compounds comprising at least two functional groups which are capable of entering into a polymerization reaction are used as the cross-linking agents. A cross-linking agent is necessary in order to maintain high deformation-strength properties of a polymer network when a large amount of collagen is introduced. The concentration of the cross-linking agent cannot be more than 10% because of an increase of the embrittlement of the material at higher values. The introduction of an UV adsorber with a concentration greater than 10% may result in a reduction of the transparency of the material.

Compounds absorbing UV radiation are used as the UV adsorbers, for example, diphenyl ketones and derivatives thereof, styrenes and derivatives thereof, etc.

### EXAMPLES

### Brief Description of Technology of Production

A UV adsorber or a mixture of UV adsorbers in a concentration in accordance with the invention is dissolved in all ethylenically unsaturated compound or mixture of such compounds, and is mixed with a collagen solution. If necessary, a cross-linking agent or a mixture of such agents in a concentration in accordance with the invention is added to the solution. The solution is filtered, degasified, and polymerization is initiated. The product is produced by forming during polymerization or by grinding from an intermediate product.

### Example 1

| | |
|---|---|
| Collagen | 20% |
| Vinylpyrrolidone | 10% |
| TGM-3* | 10% |
| Meta-vinyl diphenyl ketone | 10% |
| Water | 50% |

*TGM-3-triethylene glycol dimethacrylate

This composition was polymerized in a rotary form for production of contact lenses.

| | |
|---|---|
| Absorption in the UV region of the spectrum | 99% |

### Example 2

| | |
|---|---|
| 2-hydroxyethyl methacrylate | 50% |
| Diethylaminoethyl methacrylate | 49.999% |
| 11-hydroxy undecyl methacrylate-methadiphenyl ketone | 0.001% |
| Intrastromal lenses were ground from the obtained material. | |
| Absorption in the UV region of the spectrum | 98% |

### Example 3

| | |
|---|---|
| Collagen 1 | 2% |
| Hydroxybutyl methacrylate | 90% |
| TGM-6** | 3% |
| TGM-13*** | 2% |
| Water | 3% |
| Intraocular lenses were ground from the obtained material. | |
| Absorption in the UV region | 60% |

**TGM-6-hexaethylene glycol dimethacrylate
***TGM-13-tridecaethylene glycol dimethacrylate

### Example 4

| | |
|---|---|
| Collagen | 10% |
| N-vinylpyrrol idone | 80% |
| 11-hydroxy undecyl methacrylate-methadiphenyl ketone | 0.01% |
| Water | 9.99% |
| Contact lenses were ground from the obtained material. | |
| Absorption in the UV region | 99% |

### Example 5

| | |
|---|---|
| Collagen | 0.00001% |
| 2-hydroxyethyl methacrylate | 78% |
| TGM-13*** | 2% |
| 11-hydroxy undecyl methacrylate-methadiphenyl ketone | 0.5% |
| Meta-vinyl diphenyl ketone | 0.19999% |
| Water | 19.3% |
| Intraocular lenses were ground from the obtained material. | |
| Absorption of lenses in UV region | 99% |

*TGM-3-triethylene glycol dimethacrylate
**TGM-6-hexaethylene glycol dimethacrylate
***TGM-13-tridecaethylene glycol dimethacrylate

1. A biocompatible optically transparent polymeric material, comprising collagen, at least one water-soluble ethylenically unsaturated compound, a cross-linking agent, and at least one UV adsorber having at least one functional group, wherein said biocompatible material has the following relationship of the components (wt. %):

| | |
|---|---|
| Collagen | 0.00001-20 |
| Ethylenically unsaturated compound | 10-99.99899 |
| Cross-linking agent | 0-10 |
| UV adsorber | 0.001-10 |
| Water | balance to 100. |
